(19) United States
(12) Patent Application Publication
(10) Pub. No.: US 2003/0203356 A1
(43) Pub. Date: Oct. 30, 2003

(54) RNase L activator-antisense complexes

(75) Inventors: Robert Silverman, Beachwood, OH (US); Hagen Cramer, Cleveland Heights, OH (US); Douglas Leaman, Whitehouse, OH (US); Paul Torrence, Flagstaff, AZ (US)

Correspondence Address: BENESCH, FRIEDLANDER, COPLAN & ARONOFF LLP, ATTN: IP DEPARTMENT DOCKET CLERK, 2300 BP TOWER, 200 PUBLIC SQUARE, CLEVELAND, OH, 44114, US

(73) Assignee: The Cleveland Clinic Foundation, Cleveland, OH (US)

(21) Appl. No.: 10/349,163

(22) Filed: Jan. 22, 2003

Related U.S. Application Data
(60) Provisional application No. 60/350,532, filed on Jan. 22, 2002.

(51) Int. Cl. A61K 48/00; C07H 21/02; C12Q 1/70

(52) U.S. Cl. 435/5; 536/23.72; 514/44

(57) ABSTRACT

The present invention relates to methods of targeting RNA and is particularly useful for inhibiting infection by RNA viruses with complexes of an activator of RNase L and an oligonucleotide that is capable of binding to the genome, antigenome or mRNAs of a target RNA (e.g. a negative strand RNA virus) to specifically cleave the genomic or antigenomic RNA strand of the target RNA (e.g. the virus). The invention in one embodiment relates to a covalently linked complex of an oligonucleotide that is capable of binding to the genomic or antigenomic template RNA strand of a negative strand RNA virus and/or binding to an mRNA of a viral protein (an “antisense oligonucleotide”) coupled to an activator of RNase L. In a preferred embodiment of the present invention, the oligonucleotide component of the complex is complementary to a region of the viral genomic RNA strand characterized by repeated or consensus sequences.

### CROSS REFERENCE TO RELATED APPLICATIONS

This application claims the benefit of U.S. provisional application serial No. 60/350,532 filed Jan. 22, 2003.

### BACKGROUND OF THE INVENTION

The present invention relates to a composition comprising an oligonucleotide complementary to a region of a genome of RNA, preferably the genome of a negative strand RNA virus (i.e. a specific target RNA), attached to an activator of RNase L (“activator-antisense complexes”) which are useful to cleave a genomic strand, antigenomic strand or mRNAs of the RNA (e.g. RNA virus). The present invention is particularly useful for treating humans infected by RNA viruses, such as Respiratory Syncytial Virus (RSV). More particularly, the invention relates to activator-antisense complexes, in which the oligonucleotide is selected to bind to regions of the RSV RNA genome that possess repeated or consensus sequences. The invention particularly relates to activator-antisense complexes, in which the oligonucleotide is selected to bind to a portion of the RNA genome that normally has little self-hybridizing secondary structure.

Negative-strand RNA viruses may be divided into two categories, the non-segmented RNA viruses, including Rhabdoviridae, Filoviridae and Paramyxoviridae, and the segmented RNA viruses, including Orthomyxoviridae, Bunyaviridae and Arenaviridae. These families of RNA viruses include the following important pathogens: parainfluenza viruses, mumps virus, measles, respiratory syncytial virus, vesicular stomatitis virus, rabies, and influenza virus, etc. These viruses share many similarities in genomic organization and structure. The genomes of negative strand RNA viruses consist of single-stranded RNA of negative polarity. The genomic RNA must be transcribed into mRNA to direct the synthesis of viral proteins in the host cell. The viral RNA-dependent RNA polymerase controls transcription and replication of the RNA genome, thus no DNA of viral origin is involved in viral replication.

Respiratory syncytial virus (RSV), a non-segmented, negative-strand RNA virus in the pneumovirus subfamily of Paramyxoviridae, is a widespread human pathogen accounting for over 1 million deaths per year worldwide. While the majority of serious cases are children from developing countries, there are estimated to be 300,000 hospitalized cases per year in the United States. It is also believed that of childhood deaths from pneumonia caused by respiratory viral infections, 62% are due to RSV.

Similar to other negative-strand RNA viruses, the RSV genomic RNA is transcribed and translated into specific mRNAs that are translated into viral proteins required for virus reproduction followed by replication of the genome. Such replication provides additional templates for transcription as well as genomic RNA for progeny virus. The single stranded RNA genome of RSV codes for ten virus-specific proteins. The negative stranded genome is packaged in a nucleocapsid and is surrounded by a lipid envelope containing two glycoproteins. One is the fusion protein which facilitates entry of RSV into cells through host membrane and viral membrane fusion.

Aerosolized ribavirin (1-b-D-ribofuranosyl-1,2,3-triazole-3-carboxamide) is the approved treatment for RSV. Ribavirin is administered as an aerosol which is inhaled. Ribavirin therapy has several limitations including minimal efficacy in clinical use, the requirement of a tent around the patient, the potential to clog ventilating units, and the observation of some teratogenicity in animal, significant side effects and high cost. Recently, another treatment has been approved for the treatment for RSV, RESPIGAM, a polyclonal antibody administered by injection.

Activator-antisense complexes (termed therein “2-5A:AS”) have been described previously. Although antisense oligonucleotides have been used as antiviral agents, e.g.: to inhibit HIV replication and to inhibit RSV infection, WO95/22553 by Kilkuskie et al., no examples of the successful use of activator-antisense complexes as an antiviral therapy have been reported.

The mechanism of action of activator-antisense complexes is different than the mechanism of action of other antisense oligonucleotides. The activator portion of the activator-antisense complexes activates RNase L and the antisense domain serves as a specific, high affinity binding site for the target RNA. The result is the selective cleavage of the target RNA by RNase L.

Physiologically, RNase L functions as part of the interferon system in restricting virus replication in cells of higher vertebrates. Interferon treatment of cells activates genes encoding 2-5A synthetases, double-stranded RNA (dsRNA)-dependent enzymes that produce 5′-triphosphorylated, 2′,5′-linked oligoadenylates (2′,5′) from ATP. Viral dsRNAs are potential activators of these enzymes. The 2′,5′A binds to and activates RNase L resulting in the general cleavage of cellular and viral RNA; thus restricting the replication of some picornaviruses.

RNase L is not specific for cleaving viral RNA. For instance, in interferon-treated, encephalomyocarditis virus infected cells, RNase L causes degradation of ribosomal RNA. Through the activator-antisense approach, RNase L is converted from a non-specific nuclease to a highly specific endoribonuclease that selectively cleaves mRNA targets. This has been demonstrated in a cell-free system from Daudi cells, a human lymphoblastoid cell line, in which a modified HIV-1 vif mRNA was targeted for cleavage by an activator-antisense complex. Subsequently, purified RNase L has been directed by an activator-antisense complex to cleave selectively an mRNA target encoding the protein kinase PKR in the presence of a nontargeted mRNA. Furthermore, in HeLa cells, the use of activator-antisense complexes, which were directed to a sequence in PKR mRNA, resulted in the ablation of PKR mRNA and enzyme activity such that the dsRNA-mediated activation of transcription factor, NF-KB was ablated. More recently, it was shown that the activation of RNase L by an activator-antisense complex results in the catalytic degradation of PKR mRNA (k._{cat} of about 7 sec⁻¹).

### SUMMARY OF THE INVENTION

The present invention relates generally to analogs of 2-5A synthesized and attached to a guide oligonucleotide (short strand of DNA); the guide nucleotide is preferably designed to recognize specific target RNA in an antisense manner in which the guide nucleotide sequence is specifically complementary to the nucleotide sequence of the target RNA. Upon delivering to the cells, the 2-5A portion binds and activates the RNase L while the guide sequence hones in on target RNA. The ribonuclease then cleaves the target RNA molecule and moves on to the next one until the target RNA molecules are substantially depleted or are no longer available. More particularly, the present invention relates to chimeric molecules comprising an oligonucleotide complementary to a region of the RNA genome, the RNA antigenome or mRNAs of a target RNA (e.g. a negative strand RNA virus) attached to an activator of RNase L (“activator-antisense complexes”) which specifically cleave a genomic or antigenomic strand of the RNA virus.

The present invention relates to methods of inhibiting infection of a negative strand RNA virus with activator-antisense complexes targeted to the RNA genome of the RNA virus. In particular, the invention relates to a complex of an oligonucleotide that is complementary to some portion of the genomic or antigenomic strand of an RNA virus, such as RSV, coupled to an activator of RNase L.

In a preferred embodiment, the present invention relates to a complex that is useful for the treatment of infection by a negative strand RNA virus, in particular infection by RSV. The invention relates to a complex of an activator of RNase L coupled to an oligonucleotide complementary to a region of the virus RNA genome characterized by repeated or consensus sequences. In particular, the oligonucleotide component of the complex has a sequence of approximately 12 to 25 nucleotides complementary to a number of repeated or consensus sequences that occur within the critical gene-end-intragenic-gene-start signals of the virus RNA genome.

In another embodiment, the present invention relates to a complex of an oligonucleotide complementary to a region of the virus RNA antigenome or mRNA coupled to an activator of RNase L. The essential components of the complex are an antisense oligonucleotide which has a sequence that is complementary to between about 10 and about 30 nucleotides of the antigenomic RNA strand, i.e., the template strand for genome synthesis, of a strain of RSV and an activator of RNase L (henceforth generally referred to as “activator-antisense complexes”). In a further alternative embodiment the invention consists of an antisense oligonucleotide having a sequence of at least 10-30 nucleotides and preferably 15-25 nucleotides, and more preferably which is 17, 18 or 19 nucleotides. The elements of the activator-antisense complex are preferably covalently linked by a linker.

In a preferred embodiment, but not by way of limitation, the activator-antisense complexes of the invention are transported across the cell membrane without the use of carriers or permeabilizing agents. Once internalized the activator-antisense complexes lead to the formation of enzyme-antisense complexes, which causes destruction of the antisense targeted RNA. To treat RSV infection the antisense complexes can be administered by inhalation of an aerosol, the same method as is used to administer ribavirin. Ribavirin and the antisense complexes of the invention can, therefore, be administered in a common pharmaceutical composition.

### BRIEF DESCRIPTION OF THE FIGURE

FIG. 1. Comparison of anti-RSV activities of RBI245, ribavirin, and other activator-antisense complexes.

### DETAILED DESCRIPTION OF THE INVENTION

The present invention relates to methods of inhibiting infection by RNA viruses with complexes of an activator of RNase L and an oligonucleotide that is capable of binding to the genome, antigenome or mRNAs of a target RNA (e.g. negative strand RNA virus) to specifically cleave the genomic, antigenomic RNA strand or mRNAs of the RNA.

The present invention relates to a covalently-linked complex of an activator of RNase L and an oligonucleotide that is capable of binding to the genomic RNA strand of an RNA virus and/or binding to the antigenomic or mRNA of the negative strand RNA virus. In accordance with the present invention, the methods and complexes of the invention may be applied to target any negative strand RNA virus, including, but not limited to, parainfluenza virus, mumps virus, rabies, and influenza virus. The invention in one embodiment relates to a complex of an oligonucleotide that is capable of binding to the genomic or antigenomic template RNA strand of a negative strand RNA virus and/or binding to an mRNA of a viral protein (an “antisense oligonucleotide”) coupled to an activator of RNase L. In accordance with the present invention, the complex of the antisense oligonucleotide and the activator of RNase L may be covalently or non-covalently linked.

In a preferred embodiment of the present invention, the oligonucleotide component of the complex is complementary to a region of the viral genomic RNA strand characterized by repeated or consensus sequences.

In another embodiment of the present invention, the oligonucleotide component of the complex is complementary to a region of the virus RNA antigenome or mRNA which are characterized by an absence of self-hybridizing secondary structure. According to the invention, the portion of the antigenome targeted by the oligonucleotide component can be determined from the sequence of the RNA antigenome and secondary structure determining algorithms such as MFOLD. A suitable portion of the antigenome is one that is normally in a single stranded conformation, e.g., forms a loop of the stem and loop secondary structure of RNA. Since in some embodiments of the present invention the antisense activator complexes are designed to target antigenomic RNA, they are also complementary to the mRNA that directs translation of the viral proteins.

In a preferred embodiment the antisense oligonucleotide is complementary to a portion of the RSV genome or antigenome that is normally single stranded. The activator is attached through a linker to either the 3′ or the 5′ terminus of the antisense oligonucleotide by a linker. In one embodiment, a blocker is attached to the 3′ terminus of antisense oligonucleotide and the linker is attached to the 5′ terminus of the antisense oligonucleotide. In an alternative embodiment the linker is attached to the 3′ end of the antisense oligonucleotide and serves as both linker and blocker. The antisense oligonucleotide is between about 15 and about 20 nucleotides in length and preferably 17, 18 or 19 nucleotides in length. Those skilled in the art will understand that oligonucleotides with high GC content can be shorter than those with low GC content.

The internucleotide phosphodiester bonds of the antisense oligonucleotide can be any bonds that are compatible with the formation of Watson-Crick base pairs with complementary RNA. These include as non-limiting examples phosphodiesters, phosphorothiodiesters, methylphosphonodiesters and methylphosphonothiodiesters, which provide for increased resistance to degradation after administration. The nucleotides of the antisense oligonucleotide can be 2′-deoxynucleotides or 2′O-methyl nucleotides.

### Determination of the Sequence of the Antisense Oligonucleotide

The antisense component of the activator-antisense complex may be complementary to either the genomic strand (negative sense strand), the antigenomic strand (positive sense strand) or mRNAs of the target RNA (e.g. the RNA virus). The activator-antisense complexes of the present invention can be designed to be complementary to either the genomic, antigenome or mRNAs of any negative strand RNA virus, including but not limited to, respiratory syncytial virus, parainfluenza virus, influenza virus, mumps virus, and rabies virus. The present invention is exemplified by oligonucleotides directed to RSV strain A2, but the invention can be practiced with any other negative strand RNA virus having a known genomic sequence. The antigenomic sequence can be derived therefrom by routine techniques. Negative strand RNA viruses have multiple genes, i.e., the virion contains the complement of the coding strand. On entry into a host cell the genome is transcribed to produce the various mRNAs encoding the viral proteins and also to produce an entire complementary RNA, i.e., the RSV antigenome, from which the genomic strands of the progeny virus are transcribed. According to the invention, the sequence of the antisense oligonucleotide is selected so that the activator-antisense complex binds to and thereby causes the catalytic destruction of the RNA virus genomic, antigenomic strand or mRNAs.

In a preferred embodiment of the present invention, the genomic strand of RSV is targeted to those conserved sequences that occur in gene-start, intragenic and gene-end signals. In a preferred embodiment, the following sequence is used as the antisense cassette of the 2-5A-antisense chimera:

| | |
|---|---|
| 5′ AAA AAT GGG GCA AAT AA3′. | (SEQ ID NO:13) |

This 17-mer targets a number of sequences that occur within the critical gene-end-intragenic-gene-start signals of the RSV genomic RNA.

In another embodiment of the present invention, the genomic strand of RSV may be targeted using any of the following sequences as the antisense cassette of the 2-5 A-antisense chimera:

| | |
|---|---|
| 5′ GAA GAT GGG GCA AAT AC 3′ | (SEQ ID NO:14) |
| 5′ AAG GGA GGG GCA AAT AT 3′ | (SEQ ID NO:15) |
| 5′ ACA CAT GGG GCA AAT AA 3′ | (SEQ ID NO:16) |
| 5′ AAC ACA GGG GCA AAT AT 3′ | (SEQ ID NO:17) |
| 5′ AAA ACT GGG GCA AAT AT 3′ | (SEQ ID NO:18) |
| 5′ AGT TGT GGG ACA AAA TG 3′ | (SEQ ID NO:19) |

Each gene of the RSV genomic RNA begins with a conserved nine-nucleotide gene-start signal, 3′CCCCGUUUA, with the exception of the L gene, which has the signal 3′CCCUGUUUUA (SEQ ID NO:20). Transcription begins at the first nucleotide of the gene-start signal. Each RSV gene terminates with a semi-conserved 12- to 13-nucleotide gene-end signal, 3′ UCAAUUNAUAUAUUUU (SEQ ID NO:21), which directs transcriptional termination and polyadenylation. Antisense oligonucleotides, in accordance with the present invention, are those that are complementary to the critical gene-start or gene-end signals required to initiate or terminate transcription.

In accordance with the present invention, the activator-antisense complexes can also be designed to target repeated or consensus sequences of the genomic strand of other negative strand RNA viruses. In this aspect of the invention, Sendai, vesicular stomatitis and influenza viral genes are transcribed from 3′ to 5′ from a single promoter at the 3′ terminus of the genomic RNA. The 3′ and 5′ termini also contain sequences required for viral replication and viral packaging. These sequences can also be targeted by the antisense oligonucleotides of the present invention to specifically target and cleave the genomic strand of the negative strand RNA genome.

Critical sequences are abstracted from the genome and illustrated in Table 1. Here it is clear that the above 17-mer antisense cassette is a perfect hybridization match for three such vital RSV genomic RNA signal sequences. Also clear is the fact that this consensus oligonucleotide antisense sequence may additionally target other critical regions with lowered but significant efficiency. For instance, the nucleotide sequence signal at the F/intragenic M2 gene start signal, has but two mismatches to the consensus antisense sequence. Moreover, one of these is a terminal mismatch which would have a smaller effect on hybrid duplex stability than a similar internal mismatch. Likewise, the signal at the NS2-intragenic-NS2 gene-start has three mismatches, but only one is of the more critical internal variety. Following this logic, the expected order of hybridization efficiency of the consensus antisense cassette 17-mer with the different listed targets would be: 1=2=4>8>3>6,7>5>>9. In addition, another possible hybridization reaction may be possible, albeit of reduced stability compared to the perfect matches alluded to above. This would involve the possibility of interaction of the 17-mer cassette with both gene-end and gene-start sequences by a looping out of mismatches occurring in the intragenic regions, such as those in sequences 5, 6, 7, and 9.

TABLE 1

RSV 5A-ANTISENSE OLIGONUCLEOTIDES TARGETING THE GENOMIC RNA

| | | |
|---|---|---|
| antisense | 5′AAA AAT GGG GCA AAT AA3′ | mis-matches |
| (SEQ ID NO:13) | | |

| | | |
|---|---|---|
| sense Seq ID | terminal | internal |

| | | | | | |
|---|---|---|---|---|---|
| 1. | 3′-leader/NS1 start | 3′UUU UUA CCC CGU UUA UU5′ | 0 | | 0 |
| | (SEQ ID NO:22) | | | | |
| 2. | NS1/NS2 gene start | 3′UUU UUA CCC CGU UUA UU5′ | 0 | 0 | |
| | (SEQ ID NO:23) | | | | |
| 3. | NS2/N gene start | 3′CUU CUA CCC CGU UUA UG5′ | 2 | 1 | |
| | (SEQ ID NO:23) | | | | |
| 4. | N/P gene start | 3′UUU UUA CCC CGU UUA UU5′ | 0 | 0 | |
| | (SEQ ID NO:24) | | | | |
| 5. | P/M gene start | 3′UUC CCA CCC CGU UUA UA5′ | 1 | 3 | |
| | (SEQ ID NO:25) | | | | |
| 6. | M/SH gene start | 3′UGU GUA CCC CGU UUA UU5′ | 0 | 2 | |
| | (SEQ ID NO:26) | | | | |
| 7. | G/F gene start | 3′U UG UGA CCC CGU UUA UA5′ | 1 | 1 | |
| | (SEQ ID NO:27) | | | | |
| 8. | F/M2 gene start | 3′UUU UGA CCC CGU UUA UA5′ | 1 | 1 | |
| | (SEQ ID NO:28) | | | | |
| 9. | L gene start | 3′UCAACA CCC UGU UUU A C5′ | 1 | 7 | |
| | (SEQ ID NO:29) | | | | |

The result of this design is that a single 2-5A-antisense chimera would be targeted, with varying degrees of efficiency, to a large number of nucleotide sequence signals that are critical for transcription of the RSV genome to yield RSV mRNAs. Such a strategy should lead to a number of disruptions in the parent RSV genomic RNA, any one of which would, according to the model of RSV transcription and replication, be sufficient to shut down virus replication.

According to the invention, the portion of the antigenome or mRNAs that normally have no self-hybridizing secondary structure can be determined by the sequence of the RNA antigenome and secondary structure determining algorithms, such as MFOLD.

Thus, in another embodiment of the invention the sequence of the antisense oligonucleotide of the invention is selected so that the antisense oligonucleotide is complementary to a portion of the RSV genome, antigenome or mRNA and will bind to it, i.e., the activator-antisense complex targets activated RNase L to the portion of the RSV antigenome or mRNA complementary to the antisense oligonucleotide. Single stranded RNA molecules have regions in which the polymer “folds back” by self hybridizing. These regions of self hybridizing duplex RNA (“stems”) are separated by single-stranded “loops” and “bubbles.” Thus, not all portions of the RSV genome, antigenome or mRNA are susceptible to binding to the antisense oligonucleotide with equal affinity and, thus, not all portions of the RSV antigenome are suitable as targets of the activator-antisense complexes.

Which portions of an RNA molecule are in stems and which are in loops or bubbles for the purposes of the invention is determined by a computer modeling program such as “FoldRNA” or “MFOLD”, which are in the public domain (e.g., through the Biocomputing Office, Biology Department, Indiana University, Bloomington, Ind.). Such programs systematically assess all possible conformations and determine the conformation that is the most thermodynamically favored, i.e., has the lowest “free energy.” Routinely, conformations that have a free energy within 5% or 10% of the optimal conformation are also determined. Most often these nearly optimal conformations are closely related to each other, for example the position of a small bubble can differ by one or two nucleotides. As used herein a RNA strand is said to be “normally single stranded” when it is single stranded in the conformation having the lowest free energy or a free energy equivalent to the lowest free energy.

The algorithm that is implemented by these programs is described in Zuker et al., 1989, SCIENCE 244:48. The number of steps needed to calculate the lowest free energy state of a polynucleotide, according to the algorithm of Zuker is proportional to the cube of length of the polynucleotide. At present, conformations of 2 KB polynucleotides can be routinely calculated while the calculations of polynucleotides that are the length of the entire RSV antigenome (≈15 KB) are burdensome.

However, because of the kinetics of the intramolecular hybridization of polynucleotides, it is unlikely that conformations involving hybridization between widely separated portions of the polynucleotide do in fact occur even if the modeling programs indicate that they would yield a lower free energy state. Thus, no practical purpose is served by calculating the thermodynamically most stable conformation of the entire RSV antigenome. Rather, for the purposes of the invention, the conformation of the RSV antigenome can be calculated using fragments that are about 1-2 KB in length. If the predicted conformation of a particular portion of the RSV antigenome is dependent upon the length or the boundaries of the nucleotide fragment that is modeled, then the modeling program of the shorter fragment, greater than 1 KB in length, and the fragment wherein the portion is located closest to the middle of the fragment is considered to be the “normally” occurring conformation.

There are several major considerations in selecting which portions of the antisense genome are suitable as targets.

1. Since the RNase L is active on single-stranded sequences and less so on double-stranded sequences, preferably there are significant stretches of non-base-paired or minimally base-paired nucleotides near the chosen RNA target sequence.

2. Since the RNase L prefers cleavage after UNp sequences, it is preferred that the single-stranded region where cleavage may occur should contain uridine. This is preferred but not essential as it has been shown that the activator-antisense complex can direct cleavage to other nucleotides. Maran et al., 1994.

3. Since cleavage occurs on the 5′-side of the RNA target sequence, it is preferred that such uridine-containing single-stranded regions should be on the 5′-side of the target sequence.

4. Since the antisense domain of the activator-antisense complex forms a double-helical complex with an RNA target sequence, it is preferable that such a targeted sequence be located in a single-stranded or predominantly singly-stranded region of the target RNA. This is due to the consideration that such complex formation is an equilibrium process, and the magnitude of association constant for the process is reduced according to the degree and stability of secondary structure within the specific target sequence.

5. For the reasons expressed in (4) above, Zuker's MFOLD algorithm is used to generate a group of plausible RNA secondary structures. A set of structures can be generated using this program which differ only slightly in energy. Typically the folding program generates secondary structures differing in increments of 0.1 Kcal/mol, and are therefore are energetically very similar.

6. Consideration of (1-5) above leads to a search for the most preferred target sequence in an RNA target. In one embodiment, this target is single-stranded throughout the entire sequence that serves as the antisense binding site as well as a region upstream on the RNA of at least 16 and preferably at least 21 nucleotides. Thus in this situation the preferred target site should be the length of the antisense domain (e.g., 18) plus 16 equals 34 nucleotide in length. Thus, a search would be made for regions in a potential target RNA for single-stranded regions at least 34 nucleotides long and more preferably at least 45 nucleotides long.

7. One additional preference in the design of the activator-antisense complex relates to the composition of the antisense oligonucleotide. Because the activator-antisense complex operates catalytically, there must exist a necessary mechanism for the dissociation of the complex from its complementary sequence in the target RNA. Thus, it is to be expected that duplexes with a large fraction of GC base pairs would undergo dissociation with more difficulty than those having a large fraction dA-rU or dT-rA pairings. This consideration would also be a preferred design consideration.

### The Structure of the Activator

Examples of the structure of the activator are described in patent publication WO94/09129, at pages 10, 45 and 46-51, which is hereby incorporated by reference. Briefly, the activator can contain at least three riboadenylate residues, linked by 2′-5′phosphodiester bonds, having a free 5′ mono-, di- or triphosphate or thiophosphate. The 5′ thiophosphate-tetra-adenylate activator (sp5′A2′(p5′A2′)₃-O—) is the preferred activator. Other activators include p5′A2′(p5′A2′)₂-O—, sp5′A2′(p5′A2′)₂-O—, and p5′A2′(p5′A2′)₃-O—.

Phosphorothioate and phosphorodithioate linkages between adenine nucleosides can be used as well as phosphodiester. The use of these linkages results in decreased degradation but also decreased activity. Beigelmann, L., et al., 1995, Nucleic Acid Research 23:3989-94. The use of a 5′-thiophosphate results in greatly improved activity and stability. Those skilled in the art appreciate that other nucleotides can be attached to the 3′hydroxyl or 2′hydroxyl of the 2′-5′tri- or tetra-adenylate without changing its activity as an RNase L activator. Thus, these embodiments are also included in the scope of the term “activator of RNase L.” Those skilled in the art will further recognize that oligonucleotides containing bases other than adenine, such as inosine at the second nucleotide (counting 5′→3′) can also be used. Those skilled in the art also recognize that non-nucleotide activators of RNase L can be used in the invention and are equivalents of nucleotide activators. As used herein the term “2-5A” refers to any nucleotide activator of RNase L and the term “activator of RNase L” refers to any activator of RNase L including 2-5A. The term 2′,5′A refers specifically to 2′,5′-linked oligoadenylates.

### The Structure of the Antisense Oligonucleotides

The antisense oligonucleotide can have any structure now known or to be developed in the antisense art. These include phosphodiesters, phosphorothiodiesters, methylphosphonodiesters and methylphosphonothiodiesters, which provide for increased resistance to degradation after administration. The nucleotides of the antisense oligonucleotide can be 2′-deoxynucleotides or 2′O-methyl nucleotides.

The preparation of modified and unmodified oligonucleotides is well known in the art (reviewed in Agrawal et al. (1992) Trends Biotechnol. 10:152-158; Agrawal in Protocols for Oligonucleotides and Analogs, Synthesis and Properties (Agrawal, ed.), Humana Press, Totowa, N.J. (1993), Chapter 20). For example, nucleotides can be covalently linked using art-recognized techniques such as phosphoramidate, H-phosphorate chemistry, or methylphosphoramidate chemistry (see, e.g., Uhlmann et al. (1990) Chem. Rev. 90:543-584; Agrawal et al. (1987) Tetrahedron. Lett. 28: (31):3539-3542); Caruthers et al. (1987) Meth. Enzymol. 154:287-313; U.S. Pat. No. 5,149,798). Oligomeric phosphorothioate analogs can be prepared using methods well known in the field such as methoxyphosphoramidite (see, e.g., Agrawal et al. (1988) Proc. Natl. Acad. Sci. (USA) 85:7079-7083) or H-phosphorate (see, e.g., Froehler (1986) Tetrahedron Lett. 27:5575-5578) chemistry. The synthetic methods described in Bergot et al. (J. Chromatog. (1992) 559:35-42) can also be used.

In a preferred embodiment of the present invention a blocker is attached to the 3′ terminus of the antisense oligonucleotide to increase resistance to degradation by endonucleases. In one embodiment of the present, a blocker is attached to the 3′ terminus of the antisense oligonucleotide and the linker is attached to the 5′ terminus of the antisense oligonucleotide. In an alternative embodiment, the linker is attached to the 3′ terminus of the antisense oligonucleotide and serves as both linker and blocker. The blocker may be selected from the group consisting of a -p3′N5′ nucleotide, a p-O-alkylamine, a p-o-hydroxylalkylamine, a sp-O-alkylamine, a Sp-O-hydroxyalkylamine, ethyl and methyl. In yet another embodiment of the present invention, the 3′ terminus of the antisense domain is altered to include a terminal inverted 3′-3′ phosphodiester linkage which considerably increases resistance to degradation by exonucleases.

In another embodiment of the present invention a variable number of backbone phosphorothioate residues can be placed at the 5′ end and/or 3′ end and/or within the antisense domain. The phosphorothioate modification to the antisense enhances the stability of the oligonucleotide. In a particular embodiment of the present invention, three phosphorothioate residues (PS linkages) can be added to both the 5′ and 3′ ends of the antisense cassette.

### The Structure of the Linker

Any linker that covalently connects an activator of RNase L and the antisense oligonucleotide and does not prevent the activator from activating RNase L can be used in accordance with the present invention. In a preferred embodiment, the linker is attached to the 3′ or 2′ terminus of a 2-5A activator. In a further preferred embodiment the linker consists of a bis-1,4-butanediol-phosphodiester which connects the 3′ or 2′ terminus of a 2-5A activator and the 5′ or the 3′ terminus of the antisense oligonucleotide. Attachment to a terminus of the antisense oligonucleotide is selected for the convenience of synthesis. Those skilled in the art appreciate that attachment to an internal 2′ hydroxyl or to a portion of the nucleotide base that is not critical to base pairing are alternative embodiments of the invention.

### Preferred Embodiments of the 2-5A Activator Antisense Complexes

In a preferred embodiment of the present invention, the structure of the activator-antisense complex RBI245 designed to target the genomic strand of an RNA virus is:

5′sp-A2′s5′A2′s5′A2′s5′A2′-p(CH₂CH₂O)₃-p-5′(AsAsAsAAUGGGGCAAAsUsAsA)ₘ03′.

The above molecule is comprised of, but not limited to, an oligonucleotide RNA component having the sequence:

5′(AsAsAsAAUGGGGCAAAsUsAsA)ₘ-3′.

where s is a phosphorothioate linkage, p is a phosphodiester linkage, 5′sp is 5′-phosphothioate, and m is 2′-O-methyl nucleotide. All deoxy nucleotides in the oligonucleotide portion of the molecule are replaced by the 2′-O-methyl nucleotides. The terminus of the oligonucleotide RNA component is attached to a linker component:

—(CH₂CH₂O)₃—

In addition, the molecules contains an activator component having the structure:

5′sp-A2′s5′A2′s5′A2′s5′A2′-3′

containing phosphorothioate internucleate bonds which stablilizes the 2-5A moiety.

### Use of the Activator-Antisense Complexes

The activator-antisense complexes of the invention may be used to inhibit infection by a negative strand RNA virus to which the activator-antisense complex is targeted, in particular RSV infection. The activator-antisense complexes of the invention can be administered to a subject having an RSV infection by any route effective to deliver the activator-antisense complexes to the epithelium of the bronchi, bronchioles and alveoli of the subject. In one embodiment the activator-antisense complexes are delivered by use of an inhaled aerosol, according to the techniques well known in the art for the delivery of ribavirin. In a further embodiment of the invention a mixture of ribavirin and an activator-antisense complex of the invention can be administered in a common pharmaceutical carrier.

In an alternative embodiment the activator-antisense complex can be administered parenterally, e.g., by intravenous infusion. When delivered by intravenous administration, the dose of activator-antisense complex can be determined by routine methods well known to pharmacologists so that the serum concentration approximates the concentration at which antiviral activity is seen in the in vitro examples described below, e.g., a concentration of about 10 μM of spA₄-antiRSV3′-3′T/(8281-8299). When delivered by aerosol administration the dose should be selected so that the tissue concentration in the lung approximates the concentration at which antiviral activity is seen in the in vitro examples.

In yet another embodiment of the present invention, the activator-antisense complexes of the present invention have utility as a diagnostic tool to determine the presence of a specific negative strand virus in a test sample. The activator-antisense complexes of the present invention further have utility as a research tool which may be employed to better understand the negative strand RNA virus life cycle.

### Synthesis of 2-5A Antisense Chimeras

The following is an example of a method to synthesize the 2-5A antisense activators or chimeras of the present invention. Any methods known to those of skill in the art may be used to substitute or modify the methods described herein.

Synthesis and Purification of Antisense Activators

Oligonucleotide Structural Types Synthesized.

The following generic oligonucleotide types may be prepared:

I. p5′A2′p(5′A2′p)₃-[O(CH₂)₄Op]₂-5′dN3′p(5′dN3′p)ₙ5′dN

II. A2′p(5′A2′p)₃-[O(CH₂)₄Op]₂-5′dN3′p(5′dN3′p)ₙ5′dN

III. dN3′p(5′dN3′p)ₙ5′dN

IV. p5′A2′p(5′A2′p)₃-[O(CH₂)₄Op]₂-5′dN3′p(5′dN3′p)ₘ5′dN3′p-3′pdN5′

V. sp5′A2′p(5′A2′p)₃-[O(CH₂)₄Op]₂-5′dN3′p(5′dN3′p)ₘ5′dN3′p-3′pdN5′

VI. .A2′p(5′A2′p)₃-[O(CH₂)₄Op]₂-5′dN3′p(5′dN3′p)ₘ5′dN3′p-3′pdN5′

VII. .sp5′A2′p(5′A2′p)₃-[O(CH₂)₄Op]₂-5′dN3′p(5′dN3′p)ₙ5′dN

VIII. p5′A2′p(5′A2′p)₃-[O(CH₂)₄Op]₂-3′dN5′(p3′dN5′)ₙp3′dN

The following procedures are illustrative of those which may be employed to synthesize the 2-5A-antisense chimeric oligonucleotides in classes I-VIII above. In general, they follow the synthetic strategy developed in Lesiak et al., 1993.

Reagents and Chemicals Employed.

For initiation of synthesis on solid support:

dA-3′-lcaa-CPG (500 Å)

5′-O-dimethoxytrityl-N6-benzoyl-2′-deoxyadenosine-3′-lcaa-CPG

dC-3′ lcaa-CPG (500 Å)

5′-O-dimethoxytrityl-N4-benzoyl-2′-deoxycytidine-3′-lcaa-CPG

dG-3′ lcaa-CPG (500 Å)

5′-O-dimethoxytrityl-N2-isobutyryl-2′-deoxyguanosine-3′-lcaa-CPG

dT-3′-lcaa-CPG (500 Å)

5′-O-dimethoxytritylthymidine-3′-lcaa-CPG

These solid supports are used to synthesize oligonucleotides with the normal 3′→5′ phosphodiester bonds. All were 1 μmole size. These DMT protected nucleosides are attached to controlled pore glass (CPG) through a succinyl group and a long chain alkyl amine (lcaa) linker are commercially available products of Applied Biosystems (Foster City, Calif.). These supports are employed in the synthesis of generic oligonucleotide types I, II, III, and VII.

dA-5′-lcaa-CPG (500 Å)

3′-O-dimethoxytrityl-N6-benzoyl-2′-deoxyadenosine-5′-lcaa-CPG

dC-5′ lcaa-CPG (500 Å)

3′-O-d imethoxytrityl-N4-benzoyl-2′-deoxycytidine-5′-lcaa-CPG

dG-5′ lcaa-CPG (500 Å)

3′-O-dimethoxytrityl-N2-isobutyryl-2′-deoxyguanosine-5′-lcaa-CPG

dT-5′-lcaa-CPG (500 Å)

3′-O-dimethoxytritylthymidine-5′-lcaa-CPG

These solid supports are obtained form Glen Research (Sterling, Va.) and are used to synthesize oligonucleotides with the reversed polarity 5′→3′ phosphodiester bonds. All were 1 μmole size. These supports are employed for the synthesis of generic oligonucleotide types IV, V, VI, and VIII.

2. Elongation of the DNA Antisense Chain.

For normal 3′→5′ phosphodiester bond oligonucleotides, a total of 500 mg of each of the following phosphoramidites (Applied Biosystems) is dissolved in the indicated amount of anhydrous acetonitrile to make a 0.1 M phosphoramidite solution:

5′-O-dimethoxytrityl-N6-benzoyl-2′-deoxyadenosine-3′-(2-cyanoethyl-N,N-diis opropyl)phosphoramidite (5.6 mL)

5′-O-dimethoxytrityl-N4-benzoyl-2′-deoxycytidine-3′(2-cyanoethyl-N,N-diisop ropyl)phosphoramidite (5.9 mL)

5′-O-dimethoxytrityl-N2-isobutyryl-2′-deoxyguanosine-3′-(2-cyanoethyl-N,N-d iisopropyl)phosphoramidite (5.8 mL)

5′-O-dimethoxytrityl-2′-deoxythymidine-3′-(2-cyanoethyl-N,N-diiso propyl)phosphoramidite (6.6 mL)

The foregoing were used in the preparation of generic oligonucleotide types I, II, III, IV, V, VI, and VII.

For the synthesis of oligonucleotides with all DNA phosphodiester bonds with reversed polarity, the following phosphoramidites may be obtained from Glen Research (Sterling, Va.).

3′-O-dimethoxytrityl-N6-benzoyl-2′-deoxyadenosine-5′-(2-cyanoethyl-N,N-diis opropyl)phosphoramidite (5.6 mL)

3′-O-dimethoxytrityl-N4-benzoyl-2′-deoxycytidine-5′(2-cyanoethyl-N,N-diisop ropyl)phosphoramidite (5.9 mL)

3′-O-dimethoxytrityl-N2-isobutyryl-2′-deoxyguanosine-5′-(2-cyanoethyl-N,N-d iisopropyl)phosphoramidite (5.8 mL)

3′-O-dimethoxytrityl-2′-deoxythymidine-5′-(2-cyanoethyl-N,N-diisopropyl)phosphoramidite (6.6 mL)

The above intermediates were employed to synthesize generic oligonucleotide type VIII.

3. Linker to Join Chimeric Domains.

The linker, (2-cyanoethyl-N,N-diisopropyl)-[4-O-(4,4′-dimethoxytrityl)butyl] phosphoramidite, is synthesized by a modification of an earlier described procedure (Lesiak et al., 1993), and a 0.1 M solution was made by dissolving 100 mg linker in 1.7 mL of anhydrous acetonitrile.

4. For Synthesis of 2′,5′-oligoadenylate Domain of the Chimera.

5′-O-dimethoxytrityl-N6-benzoyl-3′-O-t-butyldimethylsilyladenosine-2′-N,N-diisopropylcyanoethylphosphoramidite (ChemGenes Corp., Waltham, Mass., cat no. ANP 5681). A 0.1 M solution is made by dissolving 500 mg of monomer in 5.0 mL of anhydrous acetonitrile.

5. Phosphorylation Reagent for 5′-Terminus of 2′,5′-oligoadenylate Domain of Chimera.

2-[2-(4,4′-dimethoxytrityl)ethylsulfonyl]ethyl-(2-cyanoethyl)-(N,N-diisopropyl)phosphoramidite (Glen Research, Sterling, Va. cat no. 10-1900-90) is used at a concentration of 0.2 M in anhydrous tetrazole/acetonitrile (ABI) for semi-automated synthesis.

6. Other Reagents.

All other DNA synthesis reagents may be obtained from Applied Biosystems Inc. which includes diluent (acetonitrile), activator solution (tetrazole/acetonitrile), capping solutions (A: acetic anhydride solution and B: N-methylimidazole solution), deblocking reagent (trichloroacetic acid solution), oxidizer (iodine solution), and tetraethylthiuram disulfide sulfurization reagent.

Tetrabutylammonium fluoride in tetrahydrofuran (Aldrich, Milwaukee, Wis.) was used to deblock the t-butyldimethylsilyl group used for protection of the 3′-hydroxyls of (2′,5′)-oligoriboadenylate domain.

### Automated Procedures for the Synthesis of Activator-Antisense Complexes

The following is an example of modified automated or semi-automated procedures to synthesize the 2′,5′-oligoadenylate antisense activators or chimeras of the present invention.

All of the chemicals are dried over P₂O₅ in vauco overnight before use. The 1 μmole deoxynucleoside-lcaa-CPG column was used.

The core (2′,5′)-oligoadenylate/antisense chimera refers to the complete 2′,5′A-antisense chimera minus the 5′-terminal monophosphate group and has three regions defined for synthetic purposes: an antisense region, a linker region, and (2′,5′)oligoadenylate region.

1 μmole scale standard synthesis cycle is used. The cycle is modified by changing the coupling time (coupling of monomer) for each different region. The monomer/acetonitrile solution is installed on the DNA synthesizer by a double change procedure to avoid contaminants. After the synthesis of each region, the column is dried completely by Argon for at least 3 min. and the synthesis cycle, trityl mode, and sequence are edited for the synthesis of next region of the desired oligonucleotide.

For preparation of the 5′-monophosphate terminating chimeras, the core oligonucleotide may synthesized with the trityl group on, and the column was dried and removed from the DNA synthesizer.

Cleavage and Deprotection

1. The oligonucleotide is cleaved from the CPG support by concentrated ammonium hydroxide/ethanol (3:1 v/v) at room temperature for 2 hours.

2. The ammonium hydroxide/ethanol solution of crude oligonucleotide is removed into a 3 mL vial and sealed tightly. The solution is incubated at 55° C. for 8 hours to remove the protecting groups on the bases.

3. The resulting ammonium hydroxide/ethanol solution of oligonucleotide is transferred to a glass tube, and cooled completely in a ice-bath. The solution is then evaporated to dryness in a speedvac concentrator and a solution of tetrabutylammonium fluoride (2 mL, 1.0 M) in THF is added, and the entire mixture is vortexed for at least 1 min. This reaction mixture was allowed to incubate at room temperature for at least 10 hours.

An equivalent volume of 0.1 M TEM (tetraethylammonium acetate) (pH 7.0) buffer is added, mixed and evaporated to half volume to remove THF. The residue is subjected to purification by HPLC.

Purification of the Oligonucleotides

1. Polystyrene Reverse-Phase Ion-Pair Chromatography (PRP-IPC) Protocol (a modification of the method of Swiderski, et al., 1994).

The oligonucleotide is dissolved in about 4-5 mL water to make a clear solution (centrifuged if necessary), and the clear solution is directly injected into the PRP-1 HPLC column (300×7 mm). The reaction mixture is thus simultaneously desalted and purified.

Solvent A: 10 mM tetrabutyl ammonium phosphate (TBAP), pH 7.5 in water.

Solvent B: 10 mM TBAP, pH 7.5 in acetonitrile/water (8:2 v/v).

The sample is eluted with a convex gradient of 5-90% solvent B in A in 60 min. at a flow rate of 1.5 mL/min.

Fractions containing desired oligo are pooled and evaporated to about 1-2 mL. The oligo-TBA ion-pair is converted into its sodium salt form by the following procedure:

1 mL of Dowex 50W ion exchange wet resin (Na⁺ form) is added into oligonucleotide/water solution. The solution is stirred for at least 30 min. in the cold room. The resin is removed by passing the solution through a Poly-Prep chromatography column (Bio-Rad, Cat. #731-1550). The resin is washed with extra water until no oligonucleotide remained on the resin.

Alternately, prior to Dowex treatment the oligonucleotide is passed through a C-18 Sep-Pak cartridge according to the following procedure.

a. The C-18 cartridge is pre-washed with 10 mL methanol and 10 mL water.

b. The oligo solution is loaded onto the cartridge.

c. The cartridge is washed with 20 mL water to remove salt from the column.

d. The oligonucleotide is eluted with 10 mL of 50% methanol in water.

e. The desalted oligonucleotide is detected by UV spectrophotometer and the fractions containing oligo are combined and concentrated.

Dialysis of (2′,5′)-Oligoadenylate/antisense Chimeras

After Purification by HPLC and ion exchange, the oligonucleotide (sodium salt) is dialyzed to remove small molecules and excess salt. The dialysis is carried out at 4.degree. C. The oligonucleotide is dialyzed against 0.02 M NaCl first for 4-6 hours and then against water for 48 hours. If the oligonucleotide is desalted on C-18 sep-pak cartridges after HPLC purification, the time of dialysis can be shortened to 6-10 hours.

Post-Treatment of Oligoadenylate/Antisense Chimeras

The oligonucleotide, after dialysis, is passed through a 0.22 μM millex-GV filter unit (Millipore, Cat. No. SLGVO25LS) for sterilization. The resulting solution is quantitated as O.D. A260 by UV/Vis spectrophotometry.

Nucleotide Comnosition Analysis of (2′,5′)-Oligoadenylate/Antisense Chimeras

1. Nucleotide Composition Analysis.

The nucleotide composition of the chimeric oligonucleotide is analyzed by enzymatic digestion with snake venom phosphodiesterase (Crotallus durissus) (Pharmacia, cat #27,0821-01).

A purified oligonucleotide (0.2 A260 O.D.U.) is incubated with snake venom phosphodiesterase (0.15 units) in 50 mM Tris/HCl, pH 8.0, 0.5 mM MgCl.sub.2, pH 8.0. The 100.mu.L mixture is incubated at 37.degree. C. for at least 3 hours. For chimeric oligonucleotides containing a 3′-3′dN, such as Oligonucleotide Structural Type IV, the incubation time was extended to 10 hours.

After digestion, the solution is filtered with a Microconcentrator-10 (Amicon, Inc. product No. 42406). The microconcentrator is first spin-rinsed with water before addition of 100.mu.L sample solution. The centrifugation time is typically 45 min. The clear solution is used for HPLC analysis.

An aliquot (5-10.mu.L) of the hydrolysate is analyzed by reverse phase HPLC using a Beckman Ultrasphere C-18 ODS column (0.46.times.25 cm). Separation of the digestion products is accomplished under the following conditions: 2% B isocratically for 20 min. linear gradient 2-50% B for 15 min. and held isocratically 10 min where solvent A was 100 mM ammonium phosphate, pH 5.5 and solvent B is methanol/water (1:1 v/v). The flow rate may be 0.5 mL/min. The standard markers dCMP, TMP, dGMP, AMP and dAMP (Aldrich Chem. Co.) may be used to compare retention times and elution orders of the hydrolysis products. Typically, the peaks obtained from the enzymatic hydrolysis of an oligonucleotide have retention times of 9.7 min. (dCMP), 27.3 min. (TMP), 29.6 min. (dGMP), 31.7 min. (AMP), 39.5 min. (Alinker) and 41.2 min. (dAMP). The retention times vary depending on the column, pH value of mobile phase and the equilibrium times of the column. The integrated peak areas provide the relative content of each nucleotide. The extinction coefficients of 7610 (dCMP), 8158 (TMP), 9969 (dGMP), 12342 (AMP & Alinker), 14361 (dAMP) measured at 260 nm in 100 mM ammonium phosphate, pH 5.5 may be used in the analysis.

Oligonucleotide Purity Confirmation

The purities of (2′,5′)-oligoadenylate/antisense chimeras may be checked by HPLC or gel capillary electrophoresis (GCE). The purity may be obtained by the integration of peak area detected at 260 nm.

1. Gel Capillary Electrophoresis (GCE) Method

The measurement of oligonucleotide purity is performed on an Applied Biosystems 270A-HT capillary electrophoresis instrument using MICRO-GEL100 (Applied Biosystems Inc.) gel filled capillaries (50 μM i.d., effective length 27 cm, running buffer, 75 mM Tris phosphate (pH 7.6), 10% methanol). Detection was at 260 nm. A typical electrophe of (2′,5′)-oligoadenylate/antisense chimera may be obtained by the following conditions: sample concentration approx. 0.1 O.D./mL, electrokinetic injection was 2 s at −5 kv. Voltage was −14 mA (19 mA) and the operation temperature are 30.degree. C. Under this condition, the (2′,5′)-oligoadenylate/antisense chimera has about 1 min. earlier elution time than that of its core analogue.

2. Dionex PA-100 Ion Exchange HPLC Method.

The purities of oligonucleotides could also be measured by a Dionex Ion exchange HPLC. Usually, the dionex PA-100 ion exchange column could provides higher resolution and better peak shape compared with other HPLC chromatographic method for the analysis of (2′,5′)-oligoadenylate/antisense chimera.

A typical chromatogram of (2′,5′)-oligoadenylate/antisense may be obtained by the following conditions: Dionex PA-100 (4.times.250 mm) column (Dionex, cat #43010). Solvent A is 25 mM Tris/HCl and 0.5% acetonitrile (pH 7.0), solvent B is 25 mM Tris/HCl, 0.5% acetonitrile and 1 M ammonium chloride (pH 7.0). The sample is eluted in linear gradient of 10-70% B in A during 30 min. and held isocratically for 10 min. at a flow rate of 1 mL/min. Detection is at 260 nm.

### EXAMPLE

### Comparative Data of RBI245

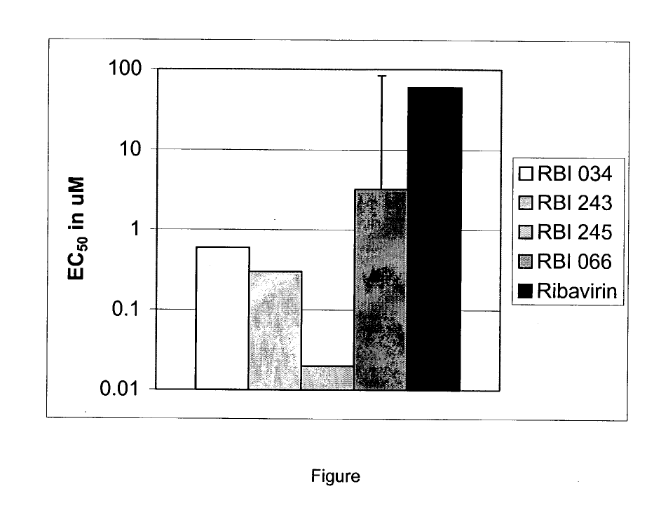

A comparison of the efficacies of RBI245 treatment and conventional ribarvirin treatment can be obtained by determining the RSV-inhibitory concentration and the cytotoxic concentration of each compound. Cultures of the human laryngeal carcinoma cell line HEp-were established and infected with an MOI=0.02. Treatment with either ribavirin or RBI245 was begun one hour post-infection. The effects of treatment on RSV infection were reported as an EC₅₀, the concentration at which there was a 50% reduction in the observable cytopathic effects of infection. The results of the treatment of HEp-2 cells with RBI245, ribavirin, and other activator-antisense complexes are shown in FIG. 1 and summarized in Table 2. HEp-2 cells treated with RBI245 had an EC₅₀ of 0.02 μM; ribarvirin had an EC₅₀ of 60 μM. Therefore, the activity of RBI245 in vitro is more than 1000-fold better than ribavarin, the only approved drug for the treatment of RSV. No toxicity was found for any of the 2-5A oligonucleotide compounds up to the maximum dosage tested (3.2 μM).

The enhanced activity of the present invention is most likely due to the stabilization of the 2-5A moiety by the phosphorothioate internucleotide bonds and the substitution of all deoxy-nucleotides in the oligonucleotide portion of the moelcule with 2′-O-methyl nucleotides.

TABLE 2

| | | | |
|---|---|---|---|
| Oligonucleotide | Structure/sequence | Description | EC₅₀ in μM* |

| | | | |
|---|---|---|---|
| RBI034 | spA₄-BU₂-(AsAsAsAAUGGGGCAAAsUsAsA)ₘ3′ | | 0.6 |
| RBI243 | (spA)₃-S9-(AsAsAsAAUGGGGCAsAsA)ₘ-3′ | shortened version of 245 | 0.3 |
| RBI245 | (spA)₄-S9-(AsAsAsAAUGGGGCAAAsUsAsA)ₘ-3′ | | 0.02 |
| RBI066 | spA₄-BU₂-(GsAsTsAGAAATAGAAAsGsCsA)ₘ-3′ | scrambled control | >3.2 |
| Ribavirin | | antiviral standard | 60 |

*50% Effective dose based on neutral red uptake assay

The present disclosure documents an approach, using 2-5A-antisense, that permits an antiviral strategy with the RSV genomic RNA (or minus RNA) as target. The 2-5A-antisense strategy has been used to target specifically repeated critical consensus regions in the respiratory syncytial virus genomic RNA. The result is a highly active 2-5A-antisense chimera, which has approximately 1000 times the anti-RSV potency of the presently employed therapeutic, ribavirin. This finding represents an innovative approach to the control of respiratory syncytial virus infection.

The present invention is not to be limited in scope by the specific embodiments described which are intended as single illustrations of individual aspects of the invention, and any constructs, viruses or enzymes which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

We claim:

1. A composition comprising:
an oligonucleotide, comprising at least one 2′O methyl nucleotide, in which the oligonucleotide is complementary to nucleotides of a genomic RNA strand, a terminus of the oligonucleotide being attached to a linker; and
an activator of RNase L attached to the linker.

2. The composition as set forth in claim 1 in which the activator is selected from the group consisting of
5′sp-A2′s5′A2′s5′A2′s5′A2′ and 5′sp-A2′s5′A2′s5′A2′.

3. The composition as set forth in claim 1 wherein the oligonucleotide is
5′(AsAsAsAAUGGGGCAAAsUsAsA)ₘ3′
wherein s is a phosphorothioate linkage and m is a 2′-O-methyl nucleotide.

4. The composition as set forth in claim 1 in which the genomic RNA strand comprises Respiratory Syncytial Virus (RSV).

5. The composition as set forth in claim 4 where the RSV comprises the A2 strain.

6. The composition as set forth in claim 1, where the nucleotides are in the conserved regions of gene-start or gene-end signals.

7. The composition as set forth in claim 1, in which the oligonucleotide comprises one or more phospho-moieties including phosphorothioate.

8. The composition as set forth in claim 1, in which the oligonucleotide consists of 2′ O-methyl nucleotides.

9. The composition as set forth in claim 1 in which the oligonucleotide is complementary to that portion of the RSV genome comprising the sequence: 3′ CCCCGUUUA 5′.

10. The composition as set forth in claim 8, in which the oligonucleotide is complementary to that portion of the RSV genome comprising the sequence: 3′ CCCCGUUUA 5′.

11. The composition as set forth in claim 1 wherein the composition is
5′sp-A2′s5′A2′s5′A2′s5′A2′-p(CH₂CH₂O)₃-p-5′-(AsAsAsAAUGGGGCAAAsUsAsA)ₘ3′
wherein s is a phosphorothioate linkage, p is a phosphodiester linkage, 5′sp is 5′-phosphothioate, and m is a 2′-O-methyl nucleotide.

12. The composition as set forth in claim 1 wherein the composition is
5′sp-A2′s5′A2′s5′A2′-p(CH₂CH₂O)₃-p-5′-(AsAsAsAAUGGGGCAAAsUsAsA)ₘ-3′
wherein s is a phosphorothioate linkage, p is a phosphodiester linkage, 5′sp is 5′-phosphothioate, and m is a 2′-O-methyl nucleotide.

13. A method of inhibiting Respiratory Syncytial Viral (RSV) infection in a mammalian cell infected with RSV which comprises a step of administering an amount of a complex effective to inhibit RSV infection, where the complex includes an antisense oligonucleotide, in which the sequence of said oligonucleotide is complementary to between 15 and 20 nucleotides of a conserved region of the genomic RNA strand of a strain of a Respiratory Syncytial Virus and a terminus of the oligonucleotide is attached to a linker; and an activator of RNase L attached to the linker.

14. A method of inhibiting Respiratory Syncytial Viral (RSV) infection in a mammalian cell infected with RSV which comprises a step of providing an amount of a complex effective to inhibit RSV infection, where the complex includes an antisense oligonucleotide, having a hydroxyl moiety at a first end, in which the sequence of said oligonucleotide is complementary to between about 15 and 20 nucleotides of a normally single stranded portion of the genomic RNA strand of a strain of a Respiratory Syncytial Virus and a terminus of the oligonucleotide is attached to a linker; an activator of RNase L attached to the linker; and a pharmaceutically acceptable, aerosolizable carrier.

15. The method as set forth in claim 13 in which the antisense oligonucleotide is 17 5′-3′-linked nucleotides.

16. The method as set forth in claim 14 in which the antisense oligonucleotide is 17 5′-3′-linked nucleotides.

17. A composition to inhibit RSV infection in a mammalian cell comprising:
an effective concentration of an oligonucleotide, wherein the oligonucleotide includes at least one 2′ O-methyl modified nucleotide, is between 15 and 20 nucleotides of complementary sequence to a conserved gene-start or gene-end signal of a Respiratory Syncytial Virus genomic RNA strand and is attached to a RNase L activator by a linker; and
a pharmaceutically acceptable carrier.

18. The composition as set forth in claim 17, further comprising a pharmaceutically acceptable, aerosolizable carrier.

19. The composition as set forth in claim 18 where the aerosolizable carrier comprises a formulation suitable for nasal administration.

20. A composition comprising an oligonucleotide complementary to a region of a virus RNA genome, a RNA antigenome or mRNA of a negative strand RNA virus linked to a Rnase L activator.

21. The composition as set forth in claim 20, wherein the oligonucleotide is complementary to between about 12 to about 25 nucleotides repeated in a gene-end or gene-start signals of the virus RNA genome.

22. The composition as set forth in claim 20, wherein the oligonucleotide is complementary to between about 10 to about 30 nucleotides of the RNA antigenome.
